(19) United States
(12) Patent Application Publication
(10) Pub. No.: US 2026/0069438 A1
(43) Pub. Date: Mar. 12, 2026

(54) Woven Stent With Improved Deployment Characteristics

(71) Applicant: MicroVention, Inc., Aliso Viejo, CA (US)

(72) Inventors: Hussain Rangwala, Villa Park, CA (US); Tao Lin, Lake Forest, CA (US); Shameem Golshan, Aliso Viejo, CA (US)

(73) Assignee: MicroVention, Inc., Aliso Viejo, CA (US)

(21) Appl. No.: 19/391,986

(22) Filed: Nov. 17, 2025

Related U.S. Application Data
(63) Continuation of application No. 16/673,862, filed on Nov. 4, 2019, now abandoned.
(60) Provisional application No. 62/755,142, filed on Nov. 2, 2018.

(51) Int. Cl. A61F 2/89; A61F 2/95; B21F 27/02; A61F 2/915

(52) U.S. Cl. CPC A61F 2/89; A61F 2/95; B21F 27/02; A61F 2002/91558; A61F 2240/001

(57) ABSTRACT

Described herein is a braided stent having have a plurality of different segments that can reduce or eliminate foreshortening and allow the proximal end of the stent to more reliably open when deployed in a curved vessel. The segments can be connected together by interlocking loops positioned at the end of each segment and can be formed of one or more radially progressing wave patterns.

### RELATED APPLICATIONS

This application is a continuation of and claims priority to U.S. Patent Application Serial No. 16/673,862 filed November 4, 2019 entitled Woven Stent With Improved Deployment Characteristics, which claims benefit of and priority to U.S. Provisional Application Serial No. 62/755,142 filed November 2, 2018 entitled Stent, both of which are hereby incorporated herein by reference in their entireties.

### SPECIFICATION

### BACKGROUND OF THE INVENTION

Stents or similar implantable devices are deployed within a vessel of a patient for various purposes, such as increasing the diameter of a narrowed vessel or to assist in the closure of an aneurysm. Two types of stents are commonly used for these treatments: woven stents and laser-cut stents. Woven stents are typically created with one or more wires that are woven or braided into a tubular structure, while laser-cut stents are typically formed by laser-cutting patterns into a metal tube.

Compared with laser-cut stents, woven stents tend to be relatively flexible which allows them to be positioned in both straight and curved portions of vessels. The relative strength of woven stents also allows them to be partially deployed, retracted back into the delivery catheter, and deployed again so that the physician can better achieve delivery to a target location. The use of different braiding patterns and wire sizes also allow a wide range of porosities to be achieved.

One disadvantage of woven stents is their tendency to foreshorten during deployment. For example, FIG. 1A illustrates a typical woven stent 10 within a delivery catheter 12 and having a compressed length 10A. As the stent 10 is deployed and radially expands, its length decreases to a radially expanded length 10B, as seen in FIG. 1B.

In many woven stents the amount of foreshortening can be significant. For example, some woven stents currently approved for medical use have compressed lengths 10A that are about twice as long as their radially expanded lengths 10B. In this respect, it can be difficult for a physician to properly determine where the “landing zone” of the stent will be, since the stent is constantly changing its length during deployment. Stent treatments that are highly sensitive to proper stent placement, such as deployment to cover the opening of an aneurysm, can therefore be difficult for a physician to achieve.

In contrast, laser-cut stents exhibit significantly less foreshortening characteristics between their radially compressed and expanded configurations. Instead of braided wires moving past each other during radial expansion/contraction, laser-cut stents typically allow portions of their structure to fold or bend inwardly. While this can make laser-cut stents much more predictable to deploy, they often lack the strength, flexibility, and retractability associated with woven stents.

Another disadvantage of woven stents is that when they are deployed within a curved portion of a vessel, their weaving pattern can sometimes prevent their proximal end from opening. The reason for this is that woven stents tend to be woven by positioning a wire back and forth along the length of the stent in a helical, over-under pattern (i.e., as the wire encounters other wires or portions of itself, it is alternately positioned over the or underneath these portions in an alternating pattern).

When the stent is deployed in a curved configuration, the curve creates pressure between various portions of the wire that overlap each other, making it difficult for these portions of wire to move relative to each other. The longitudinal, helical positioning of the wires imparts the force of these wires against each other down to the proximal end of the stent. Without the ability of the wires to easily move relative to each other, any unexpanded portion of the stent may have difficulty expanding.

This problem is further compounded by the fact that most approved stents can only be retrieved or recaptured by the delivery catheter until about 75% of their length is deployed. After further deployment, the stent is unlikely to be retrievable. Therefore, it is possible that a woven stent may be irretrievably deployed with a closed proximal end in some curved vessels. In contrast, a laser-cut stent does not have braided, overlapping wires and therefore is not susceptible to this proximal deployment difficulty when delivering within a curved target vessel.

Therefore, an improved woven stent is needed that overcomes the above-discussed deployment disadvantages.

### SUMMARY OF THE INVENTION

Generally, the present invention is directed to several different self-expanding woven stent embodiments that radially expand with minimal foreshortening and/or that more reliably open at their proximal end when deployed in a curved vessel.

In one embodiment of the present invention, a stent includes a plurality of adjacent stent regions that are each composed of a radially woven wire. For example, each stent portion may be composed of a wire forming a plurality of waves and that are connected to adjacent portions with an elongated portion of the wire.

In another example, each stent region forms a first set of waves and an overlapping second set of waves that are positioned opposite of the first set. The peaks of each stent region (i.e., the wire forming the peaks) are woven through or interlocked with the peaks from adjacent stent regions, connecting these regions together around their edge. The interlocked peaks of the stent regions are sized and positioned such that radial expansion of the stent regions do not substantially cause one stent region to pull another stent region in a longitudinal direction, thereby substantially preventing foreshortening of the stent during radial expansion.

In another example, a stent includes a main body region that is woven from one or more wires positioned in a longitudinal and helical location and being braided in an over-under pattern. A proximal end of the main body region includes a second stent region that is formed from one or more wires that are radially woven into one or more sets of wave shapes. This second stent region helps reduce foreshortening during deployment of the last portion of the stent and helps decouple any wire-on-wire stress created at other portions of the stent from deployment in a curved vessel.

In another example, a stent includes a plurality of separately woven stent portions that are each connected to each other via interlocking end loops. These stent portions can each be woven with one or more wires extending longitudinally helically in an over-under weaving pattern. Since the wires that make up each of the stent portions do not extend the full length of the stent, they are less likely to impart or communicate forces or friction created between overlapping wires when the stent is deployed in a curved vessel.

### BRIEF DESCRIPTION OF THE DRAWINGS

These and other aspects, features and advantages of which embodiments of the invention are capable will be apparent and elucidated from the following description of embodiments of the present invention, reference being made to the accompanying drawings, in which:

FIG. 1A is a side view of a stent in a radially compressed configuration within a delivery sheath.

FIG. 1B is a side view of the stent from FIG. 1 in a radially expanded and foreshortened configuration.

FIG. 2 is a side view of a stent with an open braid pattern according to the present invention.

FIG. 3A illustrates the wave-like, open braid pattern of the stent of FIG. 2 according to the present invention.

FIG. 3B illustrates an alternate wave-like, open braid pattern for a stent according to the present invention.

FIG. 3C illustrates another alternate wave-like, open braid pattern for a stent according to the present invention.

FIG. 4 illustrates a side view of a stent with a double wave-like pattern according to the present invention.

FIG. 5 illustrates a perspective view of the stent of FIG. 4 according to the present invention.

FIG. 6 illustrates a side view of a stent with a double wave-like pattern according to the present invention.

FIG. 7 illustrates the stent of FIG. 6 in a curved position according to the present invention.

FIG. 8 illustrates another view of the double wave-like pattern of the stent of FIG. 6 according to the present invention.

FIG. 9 illustrates a mandrel with a wire woven in the double-wave pattern of the stent of FIG. 6 according to the present invention.

FIG. 10 illustrates an enlarged view of the peaks of the stent pattern in from the stent of FIG. 6 according to the present invention.

FIGS. 11A and 11B illustrate an enlarged view of a cell of the stent of FIG. 6 illustrating a foreshortening calculation.

FIG. 12 illustrates another embodiment of a stent formed from double-wave pattern regions according to the present invention.

FIG. 13 illustrates another embodiment of a longitudinally woven stent having a radially woven end portion according to the present invention.

FIG. 14 illustrates another embodiment of a stent having a plurality of separately braided stent portions that are connected together via interlocking end loops.

FIG. 15 illustrates another embodiment of a stent having a plurality of separately braided stent portions that are connected together via interlocking end loops.

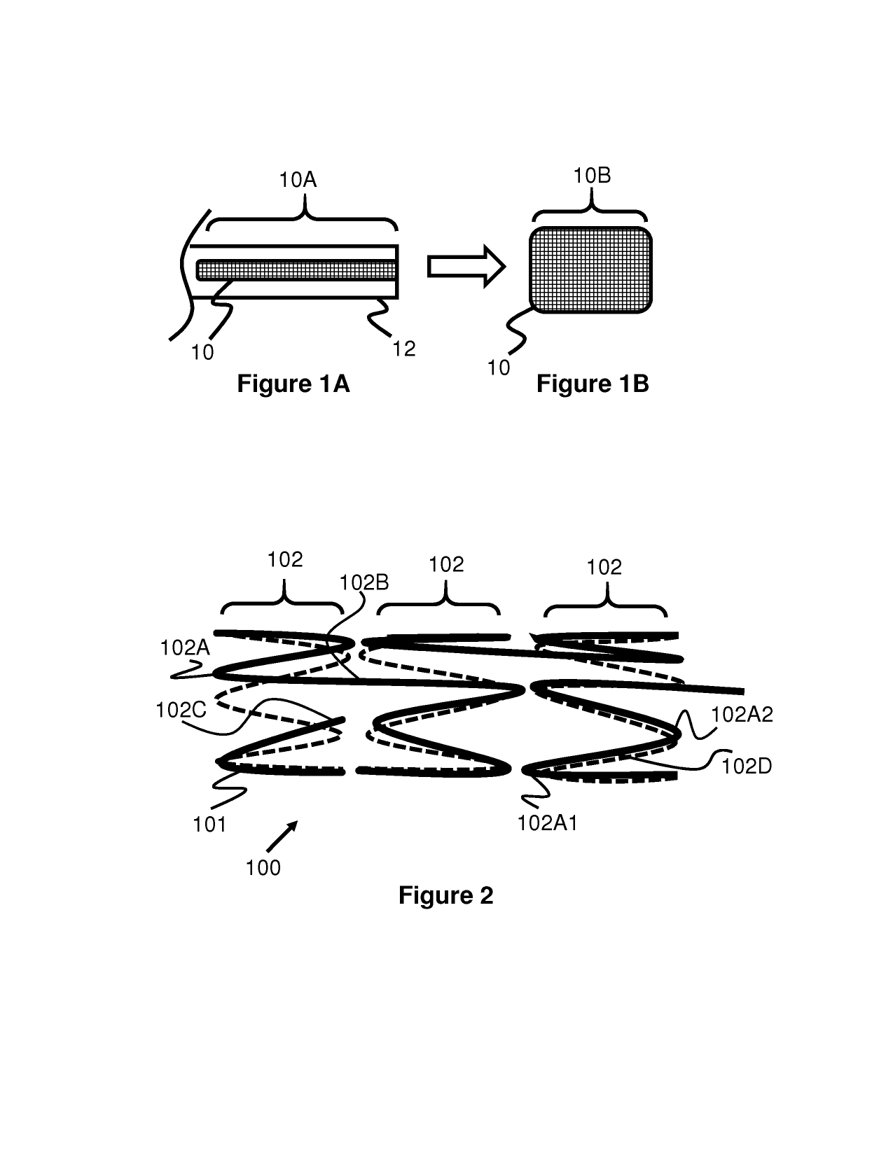

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. In that respect, elements and functionality of one embodiment are not necessarily only limited to that embodiment and may be combined with other embodiments shown herein in any manner that would result in a functional embodiment. The terminology used in the detailed description of the embodiments illustrated in the accompanying drawings is not intended to be limiting of the invention. In the drawings, like numbers refer to like elements, including between different embodiments.

Typical woven stents utilize one or more wires that are wound in a longitudinal manner around a mandrel along the entire length of the stent, creating a number of wire crossing points along the length of the stent. These wire crossing points generally leave diamond shape openings or pores along the length of the stent, representing the area between the various wire crossing locations. Due to the large number of wire crossing points over the length of the stent, the stent can be considered as one contiguous structure as opposed to a plurality of connected sections. This means there is little independence of movement across the length of the stent, which accounts for the significant foreshortening associated with typical woven stents. The wire crossing-points also can create a chokepoint at the proximal end of the woven stent, as discussed in the background section above, which can contribute to poor opening at a proximal end of the stent in particular circumstances such as deployment in a tortuous vessel.

Though traditional woven stents are considerably more flexible than laser-cut stents, these issues highlighted above can make braided or woven stents difficult to use. The present invention addresses these issues by utilizing a different stent design to maintain the flexibility of woven stents while also minimizing foreshortening and improving expansion characteristics during deployment.

Generally, the present invention is directed to woven stents that have reduced foreshortening and more reliable opening of a proximal end of the stent in curved regions as compared with currently approved woven stent designs. The present invention is also directed to methods of making and deploying these stents.

As discussed in greater detail below, the stents of this specification reduce foreshortening between their radially compressed and radially expanded positions by forming a plurality of proximally and distally facing peaks formed by a circumferentially-oriented wire wound in a sinusoidal or wave-like pattern as opposed to a helical, longitudinal direction of most traditional stents (i.e., most traditional stents weave wires in a direction along an axis of the stent). The stent according to the present invention radially expands or compresses by increasing or decreasing the bend angle of each of the peaks instead of relying on longitudinal movement of the wires in helical, longitudinally woven stents, and therefore substantially maintains a constant length.

Additionally, these stents can include a plurality of discrete segments that are connected together in a non-woven manner. These segments help isolate tension between overlapping wires that may be created when deploying in a curved vessel, ensuring that the proximal end of a stent reliably opens after being deployed.

FIG. 2 illustrates a stent 100 utilizing an open-cell interface with a sinusoidal/wave-like weaving pattern that forms a plurality of distinct stent regions 102. Note that the dashed line in this figure represents the structures of the stent 100 on the opposite side of the viewer, while the solid lines represent the structures immediately facing the viewer. Stent 100 is formed from winding a wire 101 into the generally wave-like or sinusoidal pattern. The wave pattern includes a plurality of peaks 102A proceeding in a circumferential manner around each stent region 102. Each adjacent peak faces in a different direction, such that, for example a first peak 102A1 faces in a first (e.g., distally-oriented) direction while an adjacent peak 102A2 faces in a second, opposed (e.g., proximally-oriented) direction, and in this way these peaks are alternating. Each distally facing peak 102A1 is adjacent to two proximally facing peaks 102A2, and vice-versa. A wire segment 102D connects each peak; these segments 102D can be thought of as peak linking segments since they comprise the portion of the wire between each peak 102A.

Unlike traditional woven stents which utilize a number of overlapping wire segments helically along the entire length of the stent, the regions 102 of stent 100 are connected to each other only via an elongated connection portion 102B. This single point of connection between adjacent regions 102 allows each of the regions 102 to expand and contract individually without further woven/braided connections that would otherwise cause additional longitudinal movement and thereby contribute further to foreshortening in traditional woven stents.

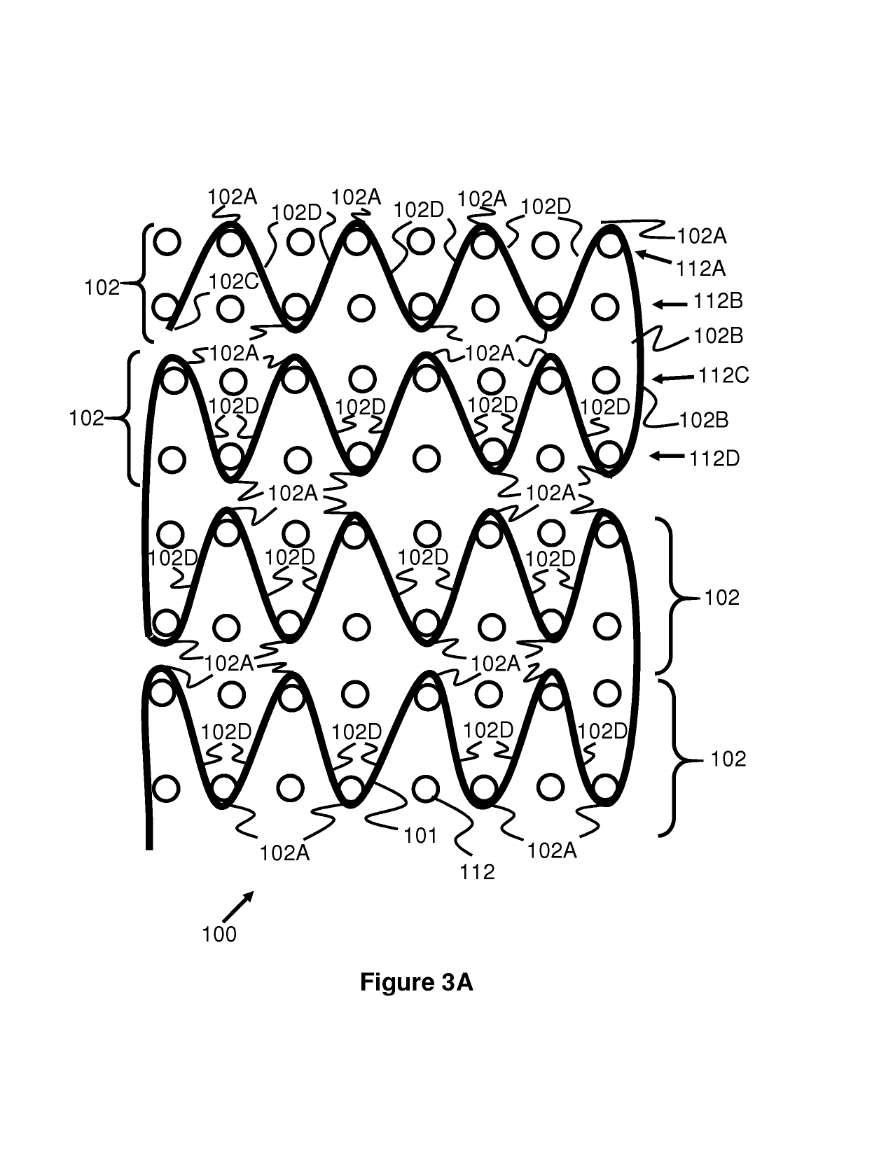

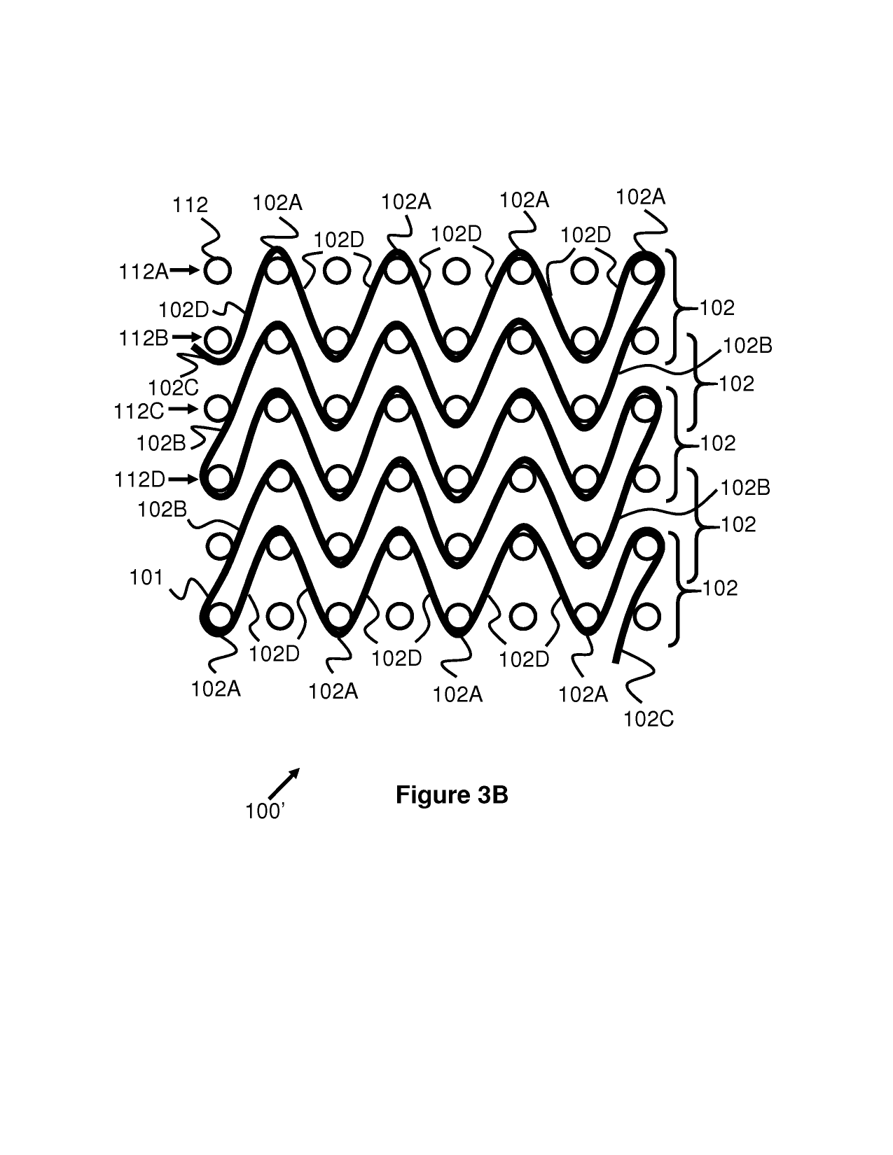

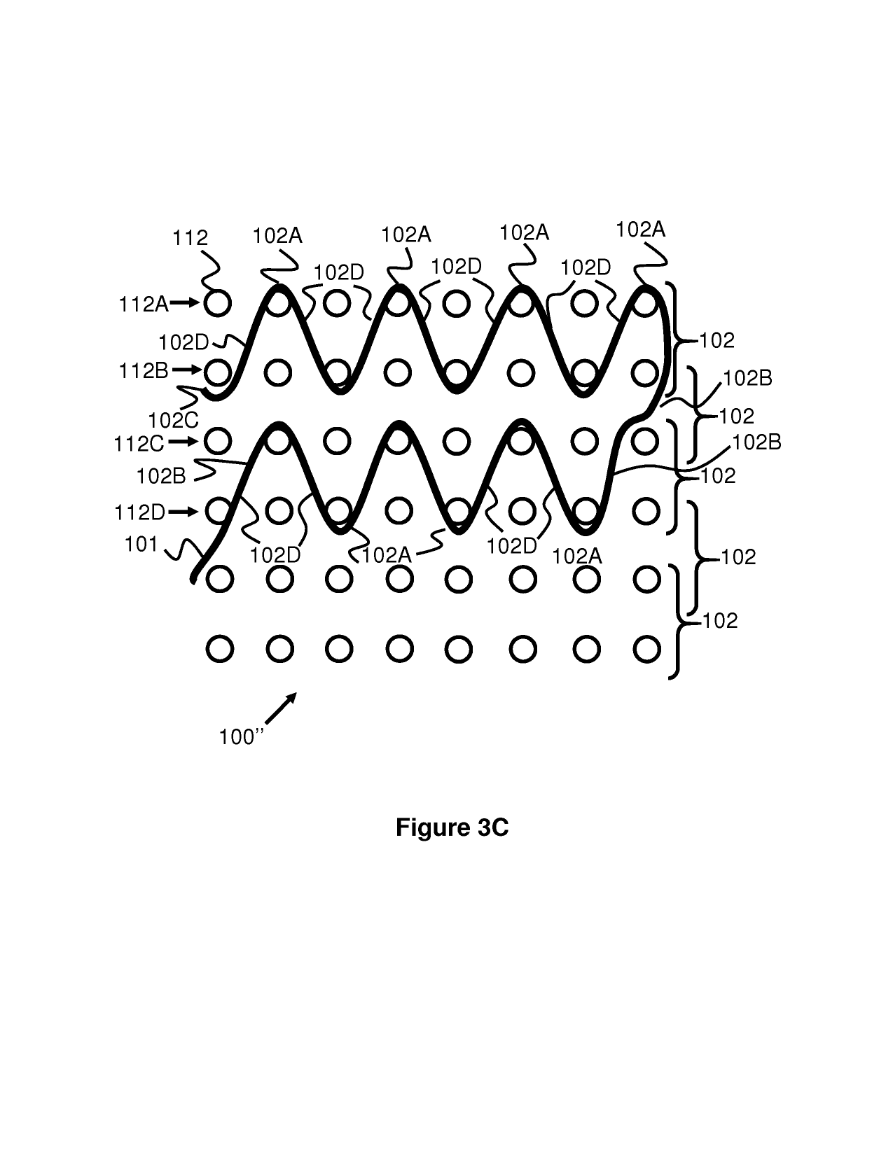

FIG. 3A illustrates the weaving pattern of one example stent region 102, as if it was flattened out in a plane. Preferably, one wire 101 is used to create the entire stent 100, though several different wires may also be used (e.g., 1 wire per distinct region 102). The wire 101 is shown woven on a mandrel having plurality of mandrel posts 112, where the wire 101 is wound around each mandrel post 112 to create a plurality of waves. Each wave comprises a peak 102A and a wound wire portion/peak linking portion 102D on each side of the peak, forming a generally sinusoidal shape. The peaks 102A alternate between a first direction and a second direction where a first peak faces in a first (e.g., distal) direction, a second adjacent peak faces in a second (e.g., proximal) direction, a third peak adjacent to the second faces in a first (e.g., distal) direction, a fourth peak adjacent to the third faces in a second (e.g., proximal) direction, and so on. In one embodiment, peak 102A has a gently curved shape. In another embodiment, the curve can be more abrupt and triangular in shape. In another embodiment, the peak 102A can have a square-like or rectangular-like shape, having two 90-degree regions separated by a flat region to create a square-shaped or rectangular-shaped wave.

The pattern of the stent 100 starts with one end 102C of the wire. While this end is shown as being a straight, terminal end, it can alternately be formed into a curved shape or loop to help prevent a loose wire end damaging a patient’s vessels (e.g., as shown in FIG. 5) or be welded to an adjacent portion of the wire 101. At the opposite end of the plurality of waves forming the first stent region 102, the wire 101 forms the elongated connection portion 102B which connects to the next adjacent stent region 102, and a second stent region 102 is then started. In one example, the elongated connection portion 102B has a length that is longer than the prior peak linking portions 102D. In one example, the elongated connection portion 102B is more than twice the length of the peak linking portions 102D and extends between a proximal end of one segment 102 and a distal end of another segment 102. Additionally, the elongated connection portion can extend relatively straight (i.e., in a direction aligned with the axis of the stent 100) to start the next segment, but can also extend at an angle similar to those of the remaining peaks 102. This longer length of elongated connection portion 102B is due to the mandrel pin placement. In the context of FIG. 3A, the first stent region 102 is wound between a first and second row of pins. The elongated connection portion 102B is then formed between the first and fourth row of pins (meaning the length is more than twice as long as the peak linking portion 102D spanning between the first row of pins and the second row of pins), and then the next stent region 102 is wound between the third and fourth rows of pins. This pattern then continues along the length of the mandrel.

This longer sizing of the elongated connection portion 102B relative to the peak linking portion 102D allows the adjacent stent regions 102 to be spaced apart from each other when expanded. This spacing helps to minimize contact between these adjacent stent regions 102, thereby promoting independent movement between these regions 102 and in turn minimizing the effects of foreshortening. In some examples, the elongated connection portion 102 has a relatively linear shape, a curved shape, or an alternating wave type of shape.

For ease of visualization, the flattened mandrel is shown as having eight rows of posts used to create a stent having four sets of wave regions 102, however fewer or more rows of posts can be used to create various numbers of stent regions 102. It should be understood that a mandrel is typically a cylindrical shape with posts 112 extending radially away from its surface. Preferably, the diameter and material of the wire 101 are selected to both hold the shape of each stent region 102 and to prevent bending or kinking of the elongated connection portion 102B during deployment or retraction. For example, for a stent 100 having an outer diameter within a range of about 2.5 to 5.5 mm (or about 0.098 inches to 0.217 inches), a wire 101 having a diameter within a range of about 0.00075 inch to 0.0035 inch can be used. Good shape-memory materials, such as nitinol can be used for wire 101 – other examples include stainless steel or cobalt-chromium. Radiopaque material (e.g. tantalum, platinum, palladium, or gold) may also be added to the stent at select locations to augment visualization of the stent.

The stent 100 is created, in one embodiment shown in FIG. 3A, by first bending an end 102C of the wire 101 around a mandrel post 112 in a first row 112A (starting on the left in the figure). The wire 101 is then positioned around a mandrel post 112 in a second row 112B that is rotationally offset (since the body of the mandrel 110 is cylindrical) from the prior post to create a peak 102A. Next, the wire 101 is positioned around a mandrel post 112 in the first row 112A that is rotationally offset from the prior post and that does not overlap itself. In this manner, only every other mandrel post 112 in each row is used, meaning one post is used in a row, the adjacent post is skipped in the same row (since the post in the row directly under is used), the next adjacent post in the same row is used, etc. This pattern is continued around the circumference of the tubular-shaped mandrel 110 until the wire 101 has been bent around every other mandrel post 112 in the first row 112A and the second row 112B. Once the wire 101 has completed its first pass around the circumference of the mandrel 110, starting from either a mandrel post 112 in the first row 112A or second row 112B (depending on how many posts 112 are in each row), the wire 101 is positioned around a mandrel post 112 in the fourth row 112D to create the elongated connection portion 102B. The wire 101 is then positioned between alternating posts 112 in rows 112C and 112D similar to the pattern in the first two rows 112A and 112B. This pattern can be continued to other rows of the mandrel. Once the pattern is completed for the entire stent 100, the stent 100 can be heat set to retain its woven shape and pattern.

Please note the description and illustrative embodiment of FIG. 3A is meant to show one particular way of winding the wire to create the sinusoidal stent pattern. Anther embodiment, shown in FIG. 3B, can utilize the elongated connection portion 102B extending between the posts of the first row 112A and the third row 112C. This means the elongated connection portion 102B is about twice as long as the prior peak linking portion 102D, since the peak linking portion 102D extends between rows 1-2 while the elongated connection portion 102B extends between rows 1-3. One example of this pattern can be seen in the stent 100’ in FIG. 3B in which an end 102C of the wire 101 is first positioned around a post 112 in the second row 112B. Next, the wire 101 is positioned around a rotationally offset pin 112 in the first row 112A and then back down to a rotationally offset pin 112 in the second row 112B to create a peak 102A. This pattern is continued around the circumference of the tubular-shaped mandrel 110 until the wire 101 has been bent around every other mandrel post 112 in the first row 112A and the second row 112B. Once the wire 101 has completed its first pass around the circumference of the mandrel 110, starting from a post 112 in the first row 112A, the wire 101 is positioned around a post 112 in the third row 112C to create connecting portion 102B. From there, the wire 101 is positioned around a rotationally offset pin 112 in the second row 112B, following a similar pattern as with the first row 112A and second row 112B. In this respect, the second stent region 102 is created with peaks 102A that align or are positioned within the peaks 102A of the first stent region 102. Put another way, the two stent regions 102 have waves that are in phase with each other, as opposed to being out of phase with each other as in the pattern of FIG. 3A. This pattern of FIG. 3B can be continued to other rows of the mandrel 110 and heat set to retain its woven shape pattern.

Furthermore, to the extent FIG. 3A shows a wind pattern where the peaks are made in such a way as to create opposed peak on each segments (e.g., a proximally facing peak facing a distally facing peak), the wind pattern can also be altered in other ways to create a pattern whereby the peaks in different rows are aligned (e.g., a proximally facing peak facing another proximally facing peak). This can be done via the technique described in the preceding paragraph. This can also be done, in the context of FIG. 3A (where elongated connection portion 102B extends between rows 1 and 4), by moving the wind pattern of the second stent segment 102 over by one pin, such that each peak is wound in an aligned manner. This is shown in FIG. 3C.

Please note, this pattern is illustratively shown as being wound on a mandrel having eight rows of mandrel pins 112, to create four stent segments 102. Where more rows are used to create a “longer” stent (e.g., 5 or more stent segments 102) the elongated connection portion 102B would simply connect between the preceding stent section 102 and the next adjacent stent section 102, meaning it would span between either the first and second mandrel pin rows, and either the third and fourth mandrel pin rows (depending on the winding configuration, as described above) where this winding pattern continues along the length of the mandrel. In this context, the elongated section 102B serves to bridge a first section 102 of the stent with the next section 102 of the stent, such that each adjacent section is connected through a distinct elongated section 102B.

In one embodiment, the stent 100 is formed on a mandrel 110 capable of creating six peaks 102A for each stent portion 102. In another example, each stent portion 102 may have 4, 5, 6, 7, 8, 9, 10, 11, or 12 peaks 102A for each stent portion 102. In one example, each peak forms an angle within a range of about 25 to 70 degrees.

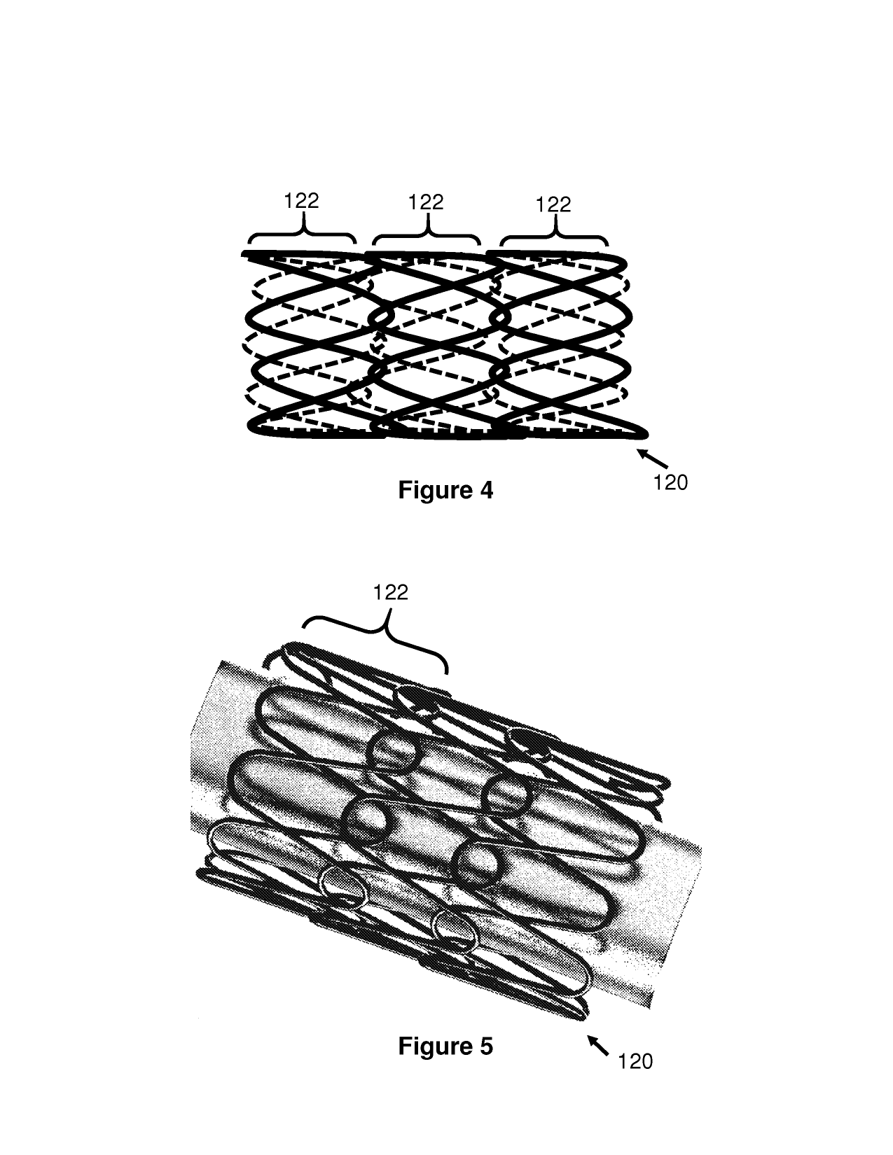

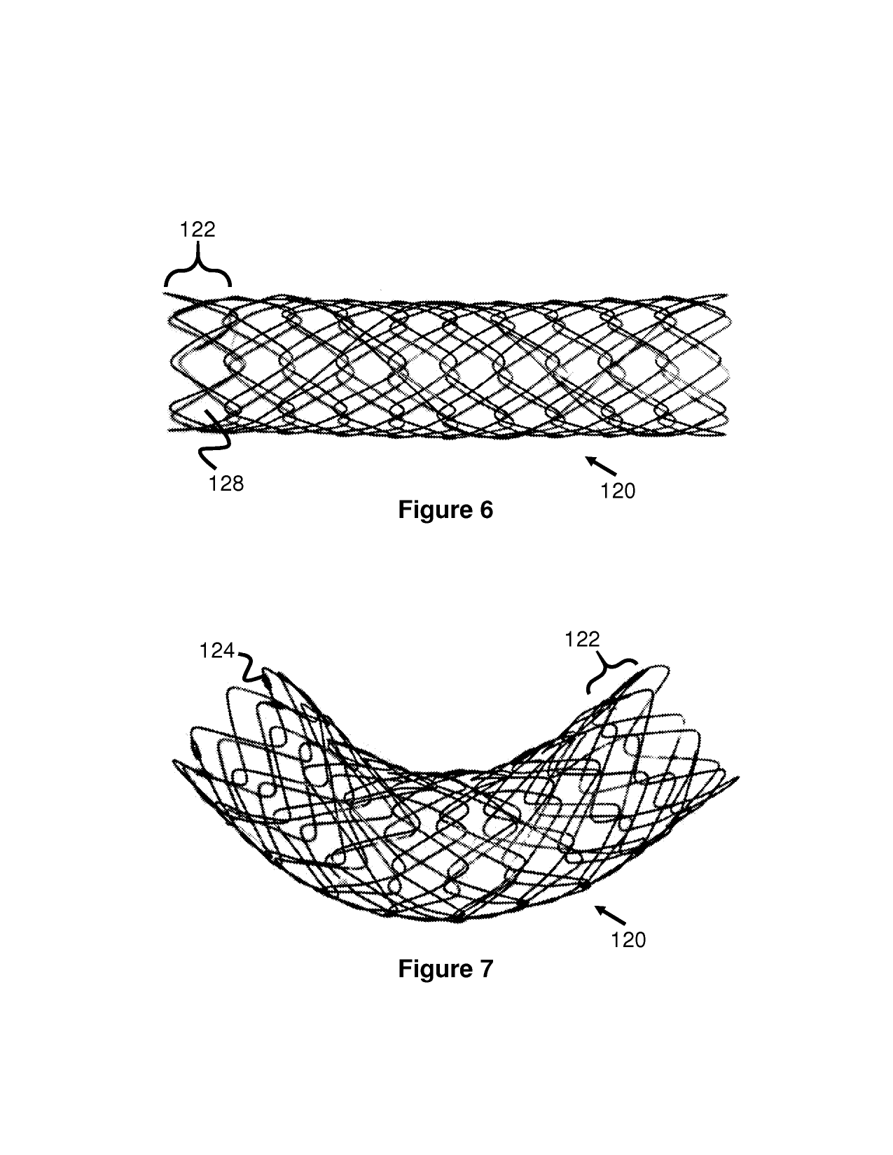

The embodiments of FIGS. 2-3C have generally shown stents utilizing independent and distinct sections 102, where each section 102 is connected to an adjacent section 102 only by an elongated connection portion 102B. FIGS. 4-7 illustrate another embodiment of a stent 120 that has a plurality of stent portions 122, but where each stent portion 122 is connected to an adjacent stent portion 122 via interlocked peaks 102A. Since each of the stent portions 122 form relatively large cells 128, it allows room for the peak 102A to move within the cell of the adjacent stent portion 122 that it is interlocked with. This interlocked configuration will be discussed in more detail later in terms of the mandrel configuration and winding process used to make the stent.

In one example, a stent 120 has a compressed configuration of about 0.017 inches in diameter and a compressed peak angle of about 0-40 degrees, or about 5-20 degrees, and an expanded configuration of about 0.157 inches in diameter and an expanded peak angle of about 10-90 degrees, or about 30-60 degrees. The compressed configuration is when the stent is in the delivery catheter, and the expanded configuration is when the stent is freed from the delivery catheter.

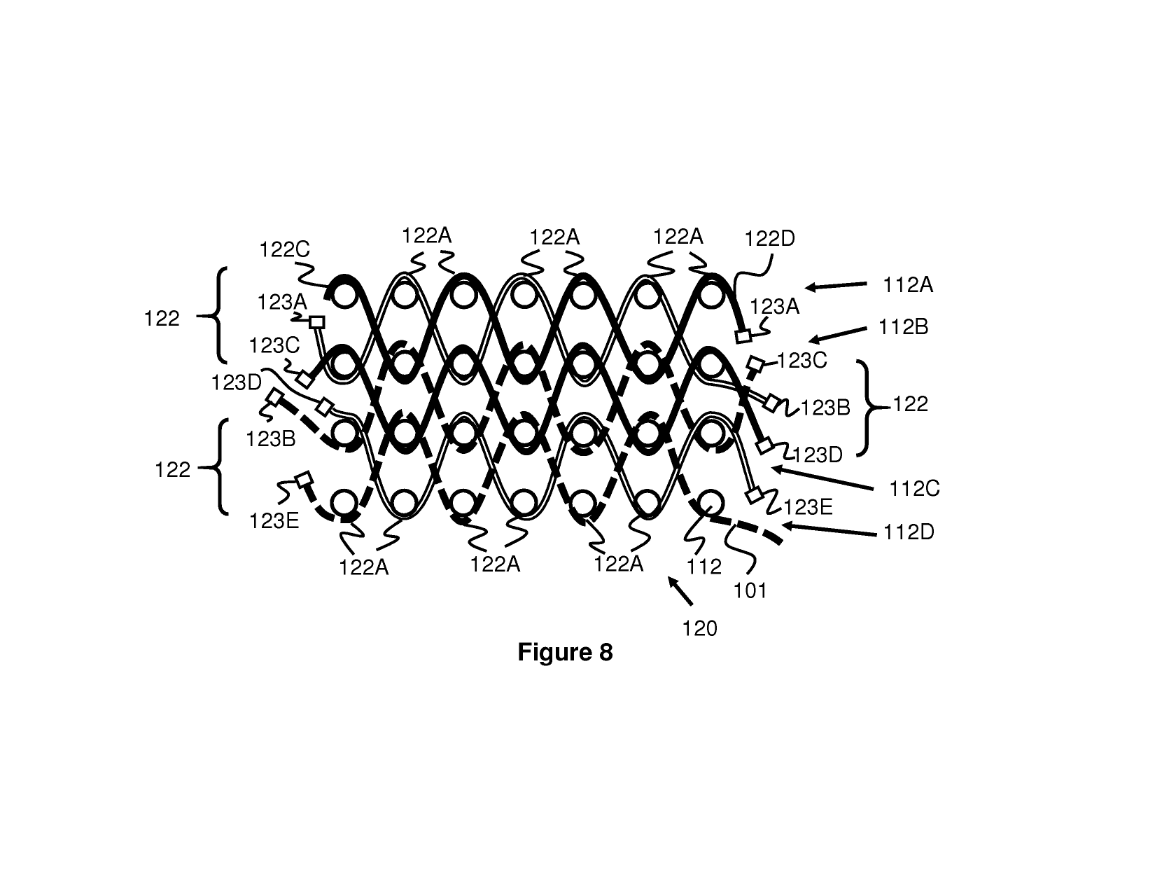

FIGS. 8, 9, and 10 illustrate a weaving pattern used to create the stent 120 of FIGS. 4-7. The stent 120 includes a plurality of stent regions 122 that each are formed of a plurality overlapping sinusoidal waves. The peaks 122A of the waves are offset from each other so that a first peak 122A is positioned in a proximal direction and a second peak 122A is positioned in opposite, distal direction. Put another way, the waves of each stent region 122 are out of phase from each other, so as to create stent cells 128. Preferably, the stent is created with only a single wire 101 woven on the mandrel 110 to create the main body (i.e., the tubular portion). However, the use of multiple wires is also possible. For example, each stent region can be woven with a different wire.

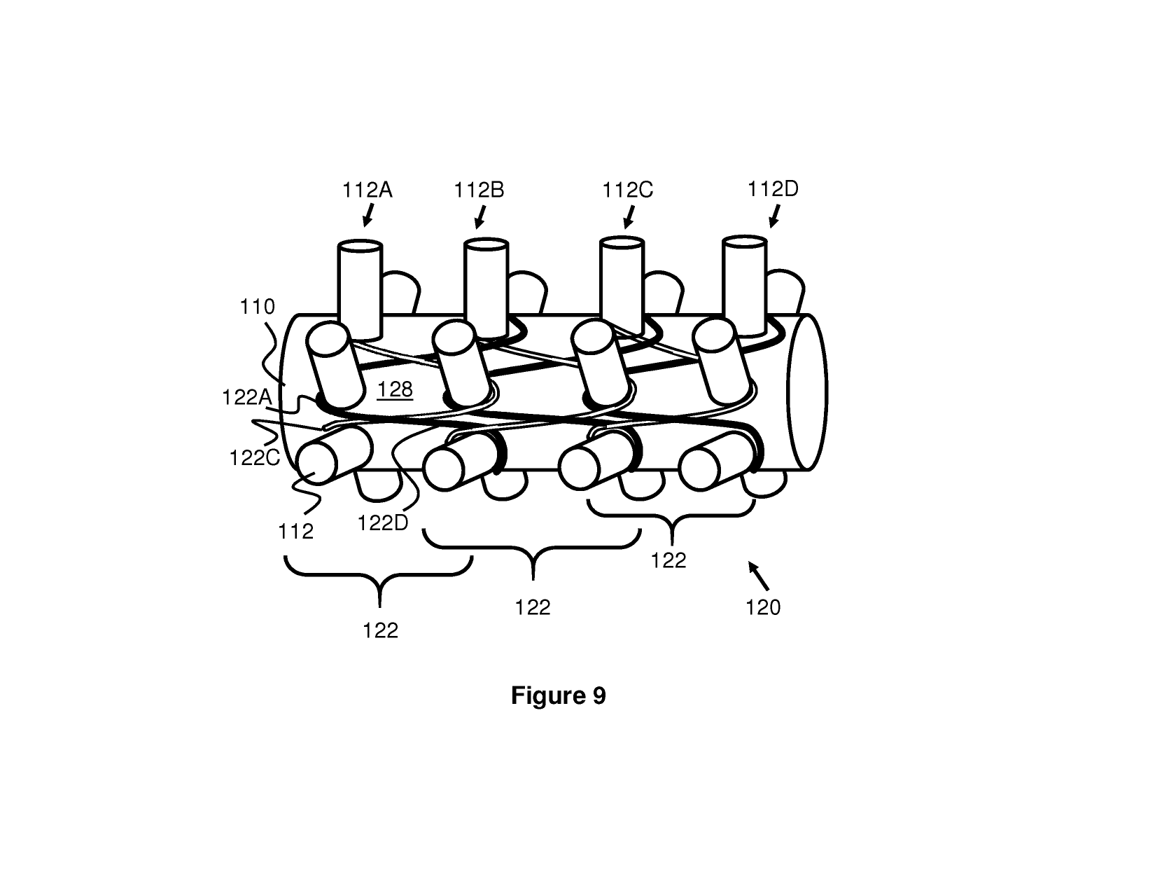

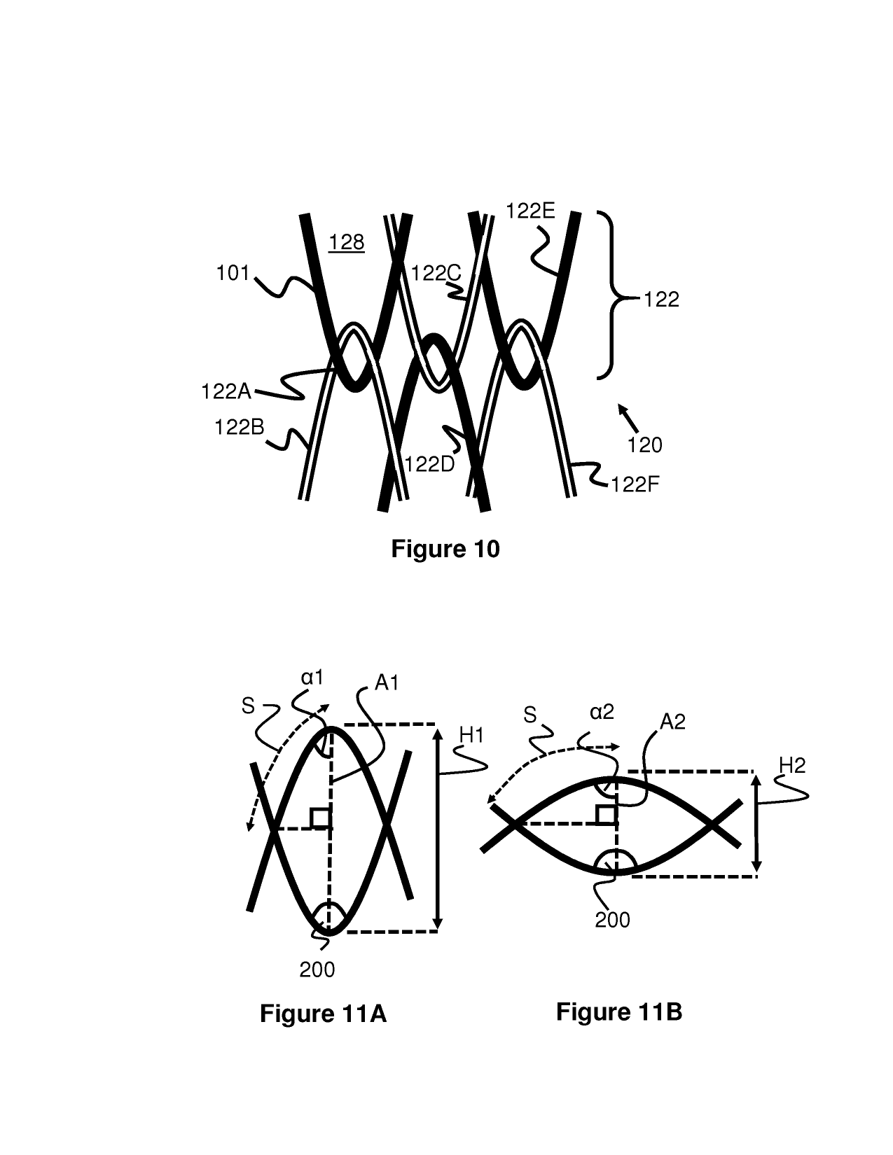

Note, FIG. 8 illustrates the weaving pattern of the stent 120 as if the mandrel that it is woven on is flattened. While one wire is preferably used for the entire stent 120, the illustrated pattern of the wire 101 is changed for each pass that the wire 101 makes around the mandrel 110 to better clarify the pattern (solid line, double line, and dashed line). Additionally, areas of the wire 101 that continue on the opposite side of the FIGURE(because the mandrel would normally be cylindrical) are designated with a square. FIG. 8 does not show the over-under pattern the wire 101 takes, but FIGS. 9 and 10 illustrate this aspect in greater detail.

The stent 120 is created, in one embodiment, by bending an end 122C of the wire 101 around a mandrel post 112 in the first row 112A (starting on the left in FIGS. 8 and 9). The end 122C can be formed into a loop or can be welded or otherwise connected to another portion of the stent region 122 once it has been created. The wire 101 is then positioned around a mandrel post 112 in the second row 112B that is rotationally offset from the prior post, to create a peak 122A. Next, the wire 101 is positioned around a mandrel post 112 in the first row 112A that is rotationally offset from the prior post without overlapping itself. This pattern is continued around the circumference of the tubular-shaped mandrel 110 until the wire 101 has passed around the circumference of the mandrel 110 once. Next, the wire 101 continues its alternating pattern between the first posts 112A and the second posts 112B (see continuation points 123A in FIG. 8), bending around the remaining “free” posts (i.e., posts that the wire 101 has not been positioned around yet). On this second pass around the mandrel 110, the wire 101 preferably alternates between being positioned over the existing wound wire/loop linking portion 122D or under the existing wound wire/loop linking portion 122D.

Once the second pass around the circumference of the mandrel 110 in the specified pattern is complete, the wire 101 has formed a stent portion 122 having peaks 122A that open toward each other (i.e., “point” in opposite directions, either proximally facing or distally facing) and wound wire segments/peak linking segments 122D between the peaks which cross each other in an alternating pattern in which the second pass of the wire 101 is positioned over a prior wire segment 122D and under a prior wire segment 122D. This alternating pattern can be seen in FIGS. 9 and 10 in which the first circumferential pass of the wire 101 around the mandrel 110 is colored white and the second circumferential pass of the same wire 101 is colored black. Due to this alternating over/under pattern, the loops are interconnected.

Next, the same wire pattern is continued between the second rows of posts 112B and third rows of posts 112C to create the next stent region 122 (see continuation points 123A, 123B, and 123C in FIG. 8). However, the wire 101 is looped through the peaks 122A formed on row 112B. This forms the interlocking peak 122A pattern best shown in FIG. 10 of two stent portions 122. Once the wire 101 has been wound for two revolutions around the mandrel 110, it is positioned further down the mandrel to proceed with the same pattern on posts 112C and 112D (see continuation points 123C, 123D, and 123E in FIG. 8). This pattern can continue to create a stent 120 of any desired length and any number of segments (e.g., 2, 3, 4, 5, 6, 7, 8, 9, 10, 11, or 12).

The mandrel 110 can have any number of posts 112 within each row (e.g., 6, 8, 10, or 12) which can affect the number of waves, weaving pattern, and similar aspects of the stent 120. Once completely woven on the mandrel 110, the stent 120 can be heat set to retain the woven shape. The free terminal ends of the wire 122C can be formed into a loop, welded, looped around adjacent portions of the wires 101, or otherwise connected to other portions of the stent 120.

In short, each stent 120 is composed of stent portions 122 in which the wire creates a first wave pattern and a second, wave pattern that is inverse or opposite of the first wave pattern, and further where each of the straight portions 122D cross each other so as to alternate between the first wave and then the second wave being on top of each other. Adjacent stent portions 122 have peaks 122A that are interlocked with the peaks 122A of immediately adjacent stent portions 122. Since these peaks are interlocked (meaning the wire comprising the peaks are intertwined due to the over/under winding pattern), the peaks cannot completely separate from each other, helping to minimize foreshortening as the stent expands from its collapsed configuration.

FIGS. 11A and 11B illustrate one cell of the stent 120 to better illustrate one example technique of calculating what the foreshortening of the stent 120 will be. While described for the stent 120, a similar calculation can be performed for stent 100, or any of the other stent embodiments with a radial sinusoidal pattern. FIG. 11A illustrates the cell of the stent 120 in a compressed configuration and FIG. 11B illustrates the cell of the stent 120 in an expanded configuration, both of which are positioned in a vertical orientation with the peaks 102/122 at their tops/bottoms similar to that shown in FIGS. 3A, 3B, and 8. With this in mind, foreshortening of the stent as a whole can be generally determined by calculating a ratio of the peak height of the stent cell in an expanded configuration compared to a compressed configuration. More specifically, the percentage that a peak decreases in height between when it’s compressed (FIG. 11A) and expanded (FIG. 11B) will represent the percentage that the stent as a whole will foreshorten. Hence, the height of the peak when compressed (H1) minus the height of the peak when expanded (H2) divided by the height of the peak when compressed (H1) represents the percent that the stent as a whole will foreshorten.

There are several ways to determine the H1 and H2 peak heights for a stent. One method is to measure the peak in both the stent’s radial expanded and compressed configurations and perform the prior calculation. A second method is to use trigonometry equations to make use of other known measurements of the peaks to provide an estimate of the height, as discussed in detail below.

As can be appreciated in the context of FIGS. 11A, when constrained inside a delivery catheter, the stent is elongated and compressed, meaning the region between adjoining peaks looks like a vertically stretched diamond having a relatively small total peak angle (200) and a relatively large total height (H1). Meanwhile, as can be appreciated in the context of FIG. 11B, the radially expanded stent will have a region between adjoining peaks resembling a horizontally stretched diamond, having a relatively large total peak angle (200) and a relatively small total height (H2). Since these regions form a diamond-type shape, trigonometrical equations can be applied to estimate the changes in the height during stent expansion.

The region or cell formed between each set of interconnected peaks resembles a diamond, as discussed above, and as can be appreciated with regard to FIGS. 11A and 11B. This diamond can be thought of as comprising four right-angled triangles. One of these right-angled triangles will have a height (A) of half the height (H) of the peak or “diamond”. The hypotenuse (S) of one of these right-angled triangles represents the length of the wire comprising half of the peak. This S value remains the same whether the stent is an expanded or compressed configuration. Even as a wire gets “wavier,” the same amount of wire is still being used since it’s simply adopting a different shape. Here, the length of the hypotenuse segment S stays the same regardless of how “wavy” the wire segment becomes since the same amount of wire is still comprising the “S” segment. Dividing the total angle of the peak in half results in the angle (α) of the right-angled triangle, as is shown with respect to FIGS. 11A and 11B. Using a trigonometrical relation, the height of the each right triangle (A1) comprising the diamond cell shape is S cos α and the total height (H) of the diamond-cell shape is twice that value, or 2S cos α. When inserted into the foreshortening ratio defined above, this becomes ((2 S cos α1) – (2 S cos α2)) / (2 S cos α1). S will be canceled out of the equation. This leaves a simplified formula of ((cos α1) – (cos α2)) / (cos α1). Hence, with only the α1 and α2 values, the foreshortening of a stent can be estimated.

Generally, this means foreshortening will be minimized in situations where peak angle and peak height do not change much as the stent adopts its expanded configurations. The sinusoidal profile can be designed in such a way to ensure this is the case, for instance by tailoring peak angle and peak height, adjusting the number of peaks in the waveform and the number of stent segments utilizing the waveforms, etc.

In one example calculation, if α1 = 2.5 degrees (5 degrees total peak angle) and α2 = 15 degrees (30 degrees total peak angle), the foreshortening is about 3.3%. If α1 = 5 degrees and α2 = 60 degrees, the foreshortening is about 13%. If α1 = 10 degrees (20 degrees total peak angle) and α2 = 15 degrees (30 degrees total peak angle), the foreshortening is about 2%. If α1 = 10 degrees (20 degrees total peak angle) and α2 = 30 degrees (60 degrees total peak angle), the foreshortening is about 12%. This means that peak angles within this range have an associated foreshortening of less than 15%, which is significantly less than traditional braided stents which can be in the range of 50%-70%. It should also be noted that the net of effect of foreshortening will tend to decrease along the length of the stent due to the cumulate impact of overlapping wires and associated friction along the stent length. Therefore, these calculations are done for a representative peak within the stent, but the overall foreshortening will likely be less than even what is calculated. Furthermore, the inclusion of more peaks in the waveform, more stent segments along the length of the stent, etc. may further reduce the overall foreshortening of the stent. Again, the foreshortening percentage should be construed as a function of the collapsed (e.g., sheathed) shape to the fully expanded shape, or in other words how much the overall length decreases as the stent expands from its collapsed delivery state to its released expanded state.

Note, these calculations have been generally described for a stent design utilizing the overlapping wire/peak concept of FIGS. 4-10. Please note, a similar calculation can be done with respect to the open-cell, non-overlapping loop concepts of FIGS. 2-3C, whereby the number of peaks, waveforms, peak angles, etc can be customized to produce a desired foreshortening. In general, its observed that for similarly sized stents, the overlapping wire/peak concepts of FIGS. 4-10 tends to decrease foreshortening due to the overlapping wire segments and all the linked waveforms. Furthermore, for similarly sized stents, increasing the number of waveforms and peaks is generally observed to decrease associated foreshortening, since each individual peak of the waveform will have less room to maneuver or change shape during expansion.

The purpose of the stent can also affect the design. Flow diversion stents are placed against the neck of a treatment site (e.g., aneurysm) and utilize a low porosity profile to reduce blood flow into the aneurysm, promoting tissue growth over time. Where a sinusoidal-type stent is used in a flow diversion setting, more wave sections 102 along the length of the stent would be preferred to increase metal surface coverage and decrease open space. In some examples, a stent with a length of about 25 mm can have about 8-15 wave sections 102. Additionally, more peaks can be used along each wave section 120 (in order words, a higher frequency/lower wavelength type wave section 102 shape) to further increase the metal surface coverage.

Coil or embolic assist stents, on the other hand, are placed against a treatment site (e.g., aneurysm) and have relatively large pore space designed to allow embolic agents such as coils to be delivered through the stent and into the target area. The stent then acts as a scaffold to keep the embolic material within the treatment site. For this purpose, more porosity is desirable. For these purposes, a stent with a length of about 25 mm can have about 5-7 wave sections 102. The height of each wave will depend on the number of wave sections used, where 5 wave-like stent sections 102 are used across the length of the stent, for a length of 25 mm each wave (representing the distance between a peak facing in one direction to the adjacent peak facing in the opposed direction) would be about 5 mm. For a similar stent length, more wave sections would result in smaller wave/peak heights while fewer wave sections would result in larger wave/peak heights. Furthermore, the number of peaks can be tailored (e.g., fewer peaks) to increase the overall porosity of the stent.

Stent 100, 120 can, in various embodiments, be wound in a number of different ways, for instance winding in such a way that each section 102/122 of the stent is wound in the same direction (e.g., clockwise) or winding in such a way that each section 102/122 is wound in an alternating direction (e.g., a first section clockwise, the next section counter-clockwise, etc). Alternating the circumferential direction (e.g., one section clockwise, the next section counter-clockwise) that the portions 102/122 are woven may introduce some resistance between sections 102/122 which may help improve the structural integrity of the stents 100/102 to remain open within a vessel after deployment. Meanwhile, a more consistent circumferential winding pattern (e.g., every section wound clockwise, or every section wound counterclockwise) would generally reduce residual stiffness of the stent and thereby improve deployment and expansion from the delivery catheter in various vessel shapes. In other words, each technique offers an advantage, depending on the particular desired characteristics of the stent. For instance, where high flexibility is needed (e.g. where the stent is deployed across a tortuous blood vessel), a consistent circumferential wind pattern can be beneficial to promote flexibility. Where high structural strength is needed (e.g., propping open a diseased vessel, diverting flow from an aneurysm, or acting as a scaffold to keep embolic material within an aneurysm), an alternating wind pattern can be used to promote structural stability.

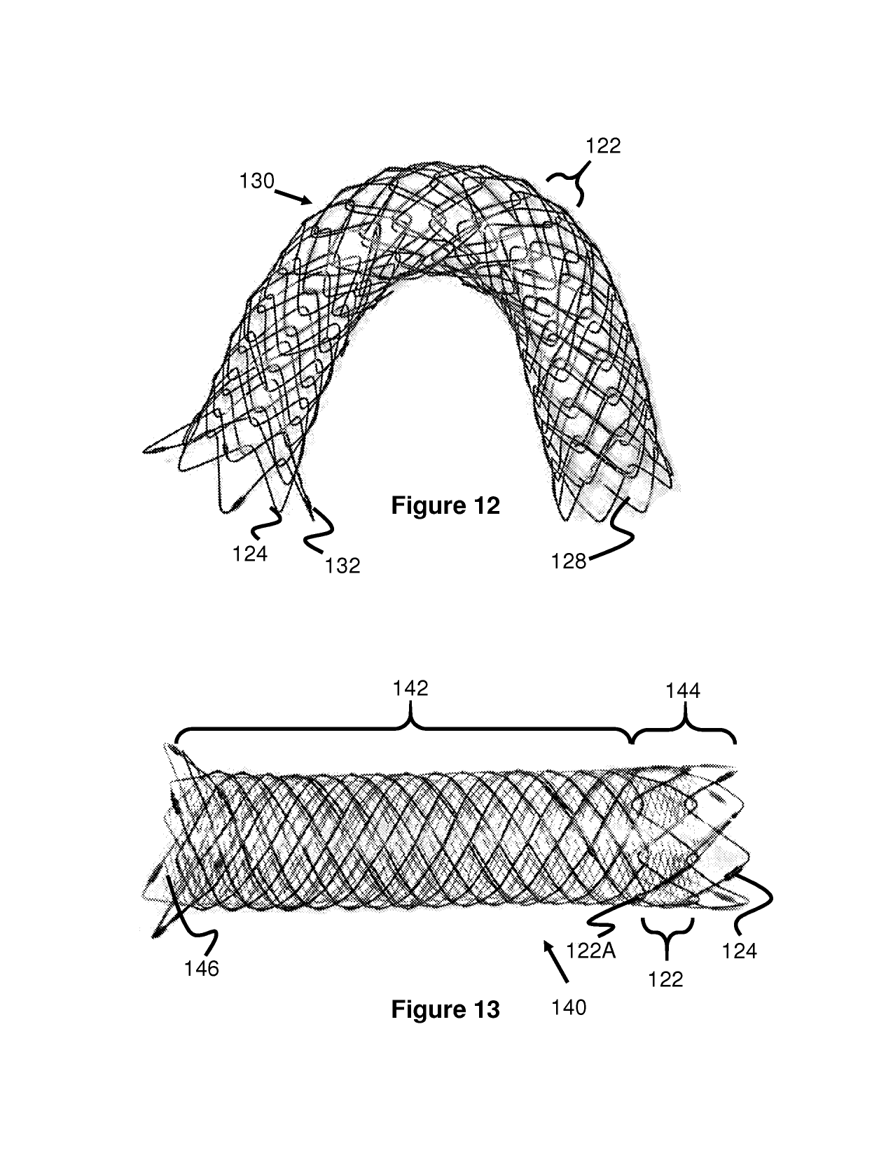

FIG. 12 illustrates another embodiment of a stent 130 that is otherwise similar to stent 120, but has at least one end with terminal loops 132 that are flared or angled radially outward relative to the remaining portions of the stent body. These flared or angled ends can help position the stent against the vessel to help resist migration. Additionally, the loops 132 may include one or more radiopaque coils 124 that can be formed by winding a radiopaque wire around the wire 101 of the stent 130. These radiopaque coils aid in imaging, and in some examples can provide a gripping interface to help mechanically grip and deliver the stent.

While the entire stent may be formed with the wavelike or sinusoidal pattern shown for stent 100 or 120, it is also possible that only smaller segments of the stent may include this pattern. For example, FIG. 13 illustrates a stent 140 having a longitudinally braided stent portion utilizing a traditional braiding technique 142 and a radially braided stent portion 144 utilizing the braided principles of the present inventive embodiments. The longitudinally braided stent portion 142 is composed of one or more wires 101 that are woven helically and longitudinally along a length of the stent 140, creating a more traditional stent weaving pattern. More details of this stent portion 142 and other aspects of a stent applicable to all of the embodiments in this specification can be found in U.S. Pat. No. 9,439,791, which is incorporated by reference. The radially braided stent portion 144 includes two wave-like stent regions 122, utilizing the wave-like winding technique utilizing interlocking peaks as discussed with the embodiments above. The peaks 122A along one edge of the braided stent portion 144 loop around or interlock with the terminal end loops of the stent portion 142. Optionally, the stent 140 may also include a braided inner layer 146 that is attached to the outer portion 142.

In this respect, the stent 140 has a portion 142 that will foreshorten as it is deployed, but also includes the sinusoidal/wave-like portion 144 that will not substantially foreshorten. While portion 144 can be positioned at either the proximal or distal end of portion 142, it may be of most use on the proximal end to allow the final positioning of the stent to be more predictably determined by the physician.

In addition to providing more predictable foreshortening, the wave-like portion 144 provides a separate braid pattern relative to portion 142 that can help decouple the proximal end from forces created by deploying in a curved vessel. This is since the two portions 142, 144 are not connected together by a single braid pattern, but instead are only connected along spaced intervals as shown in FIG. 13. As previously discussed, when deploying in curved vessels, portions of the wire press against each other and that force can travel to the proximal end of the stent, preventing the proximal end from opening. Since many stents cannot be retrieved when deployed beyond 75% of their length, it can be difficult to determine if a stent’s proximal end will fully open once deployed. By connecting a portion 144 with a different/separate braid pattern to the main portion 142, the proximal end of the stent 140 is mostly decoupled from the wire-against-wire forces created by deploying in a curved vessel. Hence, the physician can have a greater degree of confidence that the proximal end will open when fully advanced out of the delivery catheter.

For example, a physician may initially deploy the distally-oriented stent portion 142 from a delivery catheter, allowing it to foreshorten. With most of the distal portion of the stent 140 is deployed in a desired position (i.e., the portion 142), the remaining proximal wave-like portion 144 will exhibit little foreshortening as it deploys, thereby making the final placement of the proximal end of the stent 140 more predictable. Additionally, if the entire portion 142 has deployed and fully expanded, the physician can be confident that proximally-oriented wave-like region 144 will also fully expand when deployed, since it is effectively decoupled from the braid pattern of portion 142.

Some embodiments may include multiple stent segments that are all traditionally woven with wires in longitudinal, helical patterns. These segments may not reduce foreshortening as much as segments with the circumferentially braided wires but may provide the previously discussed “decoupling” effects that allow a proximal end of a stent to reliably open when deployed within a curved vessel.

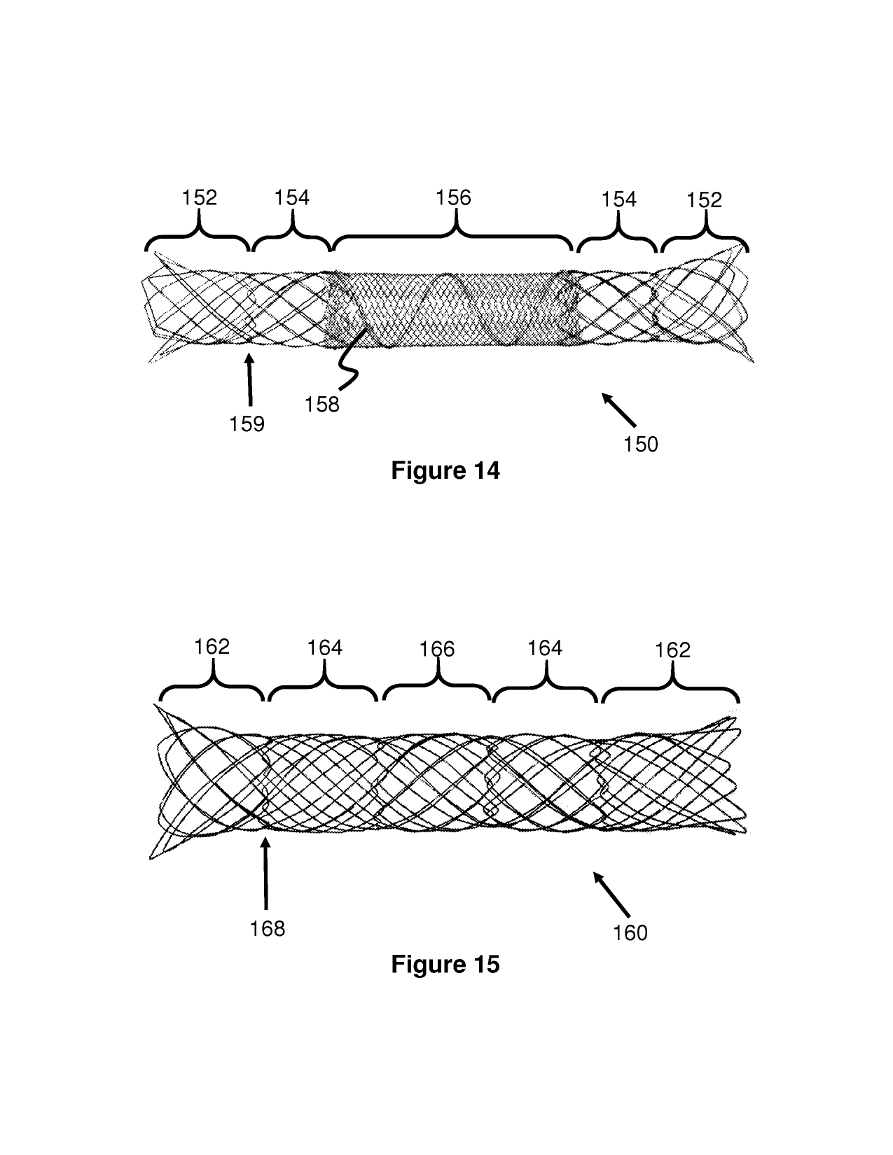

For example, FIG. 14 illustrates a stent having a woven middle portion 156, an intermediate woven portion 154 connected to each end of the middle portion 156 (i.e., two intermediate woven portions 154 total), and a terminal portion 152 connected to each end of the intermediate portion 154 (i.e., two terminal portion 152 total). Portions 152 and 154 are both woven with a wire 101 that is positioned back and forth along their length in a helical, “over-under” pattern. Portions 152 have terminal loops that are positioned through terminal loops of portion 154, creating an interface 159 that helps decouple the two woven portions from imparting strain on each other caused by deploying the stent 150 in a curved vessel.

In one embodiment, the stent 150 is created by first weaving the middle portion 156 on a tubular mandrel utilizing a traditional helical/longitudinal winding technique to create a singular mesh element. In the present example, a relatively smaller wire size, such as in the range of 0.0005 to about 0.002 inch in diameter, can be used. The wire is woven in an over/under pattern helically and longitudinally to form the length of the middle portion.

Next, the intermediate woven portions 154 are woven. These portions 154 can be further woven on the same mandrel if sufficient space is provided or can be separately woven in the traditional back and forth pattern and attached to each end of the middle portion 156 (e.g., via welding, coils, or similar methods). In the present example, the wires of each portion 154 are woven through one or more of the cells of the middle portion 156 and then are further woven in an over/under pattern helically and longitudinally to form a length of each of the intermediate portions 154. Each intermediate portion 154 can be woven from a single wire having an example diameter size in the range of 0.001 inch through 0.010 inch, though weaving with a plurality of wires of that size is also possible.

Next, the terminal portions 152 are woven. These portions 152 can be further woven on the same mandrel as the prior portions if sufficient space is provided but can be separately woven and attached also. In the present example, the wires of each portion 152 are woven through one or more of the end loops on the free end of the intermediate portion 154 and then are further woven in an over/under pattern, in which the wire is helically and longitudinally positioned to form a length of each of each portion 153. Each terminal portion 152 can be woven from a single wire having an example diameter size in the range of 0.001 inch through 0.010 inch, though weaving with a plurality of wires that size is also possible. Once this weaving is complete, the end loops of the portion 152 facing the middle portion 156 are connected to the end loops of portion 154 facing away from the middle portion 156 so as to create an interface 159 or joint within the stent 150. The stent can be deployed in a manner similar to other stents in this specification, especially in a curved vessel.

FIG. 15 illustrates another embodiment of a stent 160 having a plurality of discrete woven stent segments that are connected to each other. Specifically, the stent 160 includes a middle portion 166, two intermediate portions 164 on each side of the middle portion 166, and two terminal portions 162 on each end of the intermediate portions 164. The stent 160 can be woven in a manner similar to that of FIG. 14, starting with the middle portion 166, then weaving intermediate portions 164 on each end of the middle portion 166, and then weaving the terminal portions 162 on each free end of the intermediate portions 164. Each portion is preferably woven in an over/under pattern, in which the wire is helically and longitudinally positioned to form a length of each of each portion. Each of the portions of the stent 160 have loops around their edges which are woven to be interlocked with each other so as to form an interface 168 between adjacent stent portions. Again, by breaking the stent 160 up into several different segments, pressure exerted on the stent wires when deploy in a curved vessel may have a greater difficulty being transmitted to proximal portions of the stent 160 during delivery, making the proximal end of the stent 160 more likely to open when the stent 160 is fully deployed.

It should be understood that different aspects of the embodiments of this specification can be interchanged and combined with each other. In other words, additional embodiments are also specifically contemplated by combining different feature from different embodiments. Therefore, while specific embodiments are shown in the Figures, it is not intended that the invention necessarily be solely limited to those specific combinations.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A woven stent, comprising:
a middle portion comprising a helical pattern, wherein the middle portion comprises a plurality of open cells; and
a first intermediate portion attached to a proximal end of the middle portion, wherein a wire of the first intermediate portion is woven through one or more of the plurality of open cells of the proximal end of the middle portion, and wherein a proximal end of the first intermediate portion comprises a plurality of first end loops.

2. The woven stent of claim 1, wherein a wire diameter size of the middle portion is less than a wire diameter size of the first intermediate portion.

3. The woven stent of claim 2, wherein the wire diameter size of the middle portion is between about 0.0005 inch and 0.002 inch.

4. The woven stent of claim 2, wherein the wire diameter size of the first intermediate portion is between about 0.001 inch and 0.010 inch.

5. The woven stent of claim 1, further comprising a first terminal portion attached to a proximal end of the first intermediate portion.

6. The woven stent of claim 5, wherein a wire of the first terminal portion is woven through one or more of the plurality of first end loops of the first intermediate portion.

7. The woven stent of claim 1, further comprising a second intermediate portion attached to a distal end of the middle portion, wherein a wire of the second intermediate portion is woven through one or more of the plurality of open cells, and wherein a distal end of the second intermediate portion comprises a plurality of second end loops.

8. The woven stent of claim 7, further comprising a first terminal portion attached to a proximal end of the first intermediate portion.

9. The woven stent of claim 8, wherein a wire of the first terminal portion is woven through one or more of the plurality of first end loops of the first intermediate portion.

10. The woven stent of claim 9, further comprising a second terminal portion attached to a distal end of the first intermediate portion.

11. The woven stent of claim 10, wherein a wire of the second terminal portion is woven through one or more of the plurality of second end loops of the first intermediate portion.

12. A woven stent, comprising:
a middle portion;
a first intermediate portion attached to a proximal end of the middle portion;
a second intermediate portion attached to a distal end of the middle portion;
a first terminal portion attached to a proximal end of the first intermediate portion; and
a second terminal portion attached to a distal end of the second intermediate portion; and,
wherein each of the middle portion, the first intermediate portion, the second intermediate portion, the first terminal portion, and the second terminal portion are woven in an over/under helical pattern.

13. The woven stent of claim 12, wherein a wire diameter size of the middle portion is less than a wire diameter size of each of the first intermediate portion and the second intermediate portion.

14. The woven stent of claim 13, wherein the wire diameter size of the middle portion is less than a wire diameter size of each of the first terminal portion and the second terminal portion.

15. The woven stent of claim 13, wherein the wire diameter size of each of the first intermediate portion and the second intermediate portion is substantially equal to a wire diameter size of each of the first terminal portion and the second terminal portion.

16. The woven stent of claim 12, wherein a wire diameter size of the middle portion is between about 0.0005 inch and 0.002 inch.

17. The woven stent of claim 16, wherein a wire diameter size of each of the first intermediate portion, the second intermediate portion, the first terminal portion, and the second terminal portion is between about 0.001 inch and 0.010 inch.

18. The woven stent of claim 12, wherein the middle portion comprises a plurality of open cells, wherein a wire of the first intermediate portion is woven through at least one of the plurality of open cells of the proximal end of the middle portion, and wherein a wire of the second intermediate portion is woven through at least one of the plurality of open cells of the distal end of the middle portion.

19. The woven stent of claim 18, wherein a proximal end of the first intermediate portion comprises a plurality of first open loops, wherein a distal end of the second intermediate portion comprises a plurality of second open loops, wherein a wire of the first terminal portion is woven through one or more of the plurality of first open loops of the first intermediate portion, and wherein a wire of the second terminal portion is woven through one or more of the plurality of second open loops of the second intermediate portion.

20. A woven stent, comprising:
a middle portion comprising a plurality of open cells;
a first intermediate portion attached to a proximal end of the middle portion, wherein the first intermediate portion comprises a plurality of first open loops;
a second intermediate portion attached to a distal end of the middle portion, wherein the second intermediate portion comprises a plurality of second open loops;
a first terminal portion attached to a proximal end of the first intermediate portion, wherein a wire of the first terminal portion is woven through one or more of the plurality of first open loops of the first intermediate portion; and
a second terminal portion attached to a distal end of the second intermediate portion, wherein a wire of the second terminal portion is woven through one or more of the plurality of second open loops of the second intermediate portion;
a joint between the first intermediate portion and the middle portion;
wherein a wire diameter size of the middle portion is less than a wire size of each of the first intermediate portion, the second intermediate portion, the first terminal portion, and the second terminal portion; and,
wherein each of the middle portion, the first intermediate portion, the second intermediate portion, the first terminal portion, and the second terminal portion are woven in an over/under helical pattern.
